(19) United States
(12) Patent Application Publication
(10) Pub. No.: US 2012/0283504 A1
(43) Pub. Date: Nov. 8, 2012

(54) Biocompatible, Magnetic Nanoparticles for Treating Glioblastomae

(75) Inventors: Regine Willumeit, Hamburg (DE); Birte Mucha, Schwarzenbek (DE); Katrin Lamszus, Hamburg (DE)

(73) Assignees: UNIVERSITATSKLININKUM HAMBURG-EPPENDORF, Hamburg (DE); Helmholtz-Zentrum Geesthacht Zentrum für Material- und Küstenforschung GmbH, Geesthacht (DE)

(21) Appl. No.: 13/509,353

(22) PCT Filed: Nov. 9, 2010
(86) PCT No.: PCT/EP2010/067139

§ 371 Date: Jul. 27, 2012

(30) Foreign Application Priority Data
Nov. 12, 2009 (EP) 09175794.8

(51) Int. Cl. A61N 2/10

(52) U.S. Cl. 600/12

(57) ABSTRACT

The present invention relates to the use of biocompatible, magnetic nanoparticles for the therapy of glioblastomae in a static magnetic field. The magnetic nanoparticles according to the invention have already been in use in the diagnostics of pathological processes for several years. According to the invention, the biocompatible, magnetic nanoparticles are used for the targeted displacement of migrating cancer cells in an external magnetic field (magneto axis), in order to make said cells accessible as a collective to surgical intervention or hyperthermia.

The present invention relates to the use of biocompatible, magnetic nanoparticles in the therapy of glioblastomas.

### PRIOR ART

The glioblastoma (Glioblastoma multiforme) is the most common malignant brain tumor in adults. Approximately 15% to 30% of all brain tumors are glioblastomas. The glioblastoma is similar in its fine tissue to the glial cells of the brain and, owing to a very poor prognosis, is staged grade IV according to the WHO classification of tumors of the central nervous system. The treatment consists of surgical reduction of the tumor mass, radiation and chemotherapy. A definitive cure cannot, however, presently be achieved. The average survival time is in the order of six months.

Glioblastomas may develop as completely new tumors (de novo) or by progressive dedifferentiation from less malignant astrocytomas. It is therefore not uncommon that treated astrocytomas recur as glioblastomas. These “secondary glioblastomas” are more likely to occur in younger patients.

A characteristic of glioblastomas is diffuse, infiltrative and very rapid growth. Short-term clinical improvement can be achieved by treating the basically always present cerebral edema with dexamethasone. Neurosurgical reduction of the tumor's main mass may slow down but not permanently prevent progression of the disease, since there are basically always individual tumor cells which have already migrated in an infiltrative manner through healthy brain tissue, thereby rendering complete removal of the tumor impossible. To extend the recurrence-free and absolute survival times, surgery is followed basically always by radiation and frequently also by chemotherapy.

It is an object of the present invention to make available novel substances to the therapy of glioblastomas. This should enable therapy to be carried out in a magnetic field for the first time.

### DESCRIPTION OF THE INVENTION

The object is achieved by biocompatible, magnetic nanoparticles for the therapy of glioblastomas in a static magnetic field. Magnetic nanoparticles mean magnetizable particles, the hydrodynamic size of which is less than 1 μm, usually less than 500 nm, and preferably is in the range from 5 nm to 300 nm, particularly preferably in the range from 50 nm to 200 nm. The core diameter is preferably from 1 nm to 300 nm, more preferably 2 nm to 100 nm, and in particular 3 nm to 50 nm. The size of the magnetic nanoparticles is thus within the size range of a protein (5 to 50 nm) or a virus (20 to 450 nm).

Suitable magnetizable particles are first and foremost metals and oxides of the eighth transition group of the periodic table of the elements. Preference is given to the material, of which the biocompatible, magnetic nanoparticles consist, being selected from iron (Fe), gadolinium (Gd) or the oxides thereof. Particularly preferred materials are magnetite or its oxidized form, maghemite.

The core of the magnetic nanoparticles is preferably enclosed by a shell which has surface-active substances adsorbed or chemisorbed to its surface. Said shell is intended to prevent the particles from being able to agglomerate or form a sediment. The shell layer increases the overall diameter of the particles, which is enlarged still further due to the water binding capacity of the shell materials. The overall diameter in an aqueous solution is therefore specified by way of a hydrodynamic diameter.

Various substances may be used as shell materials of the nanoparticles. However, for medical application, they must be biocompatible to humans. Preference is given to using as shell materials polymers such as dextran, carboxydextran, polyethylene glycol, starch or albumin. If biomimetic monomers such as lipids, fatty acids, citrate, myristic acid or lauric acid rather than polymers are chosen as shell, it is possible to produce even smaller particles.

Particular preference is given to naturally occurring, biocompatible, magnetic nanoparticles such as magnetosomes from magnetotactic bacteria which are capable of synthesizing intracellular, membrane-enclosed particles from magnetite. More specifically use is made of magnetosomes from Magnetospirillum gryphiswaldense MSR-1, Magnetospirillium magnetotacticum, Magnetospirillium spec. AMB-1, magnetic coccus MC-1 or magnetic Vibrio MC-1. Magnetotactic bacteria are known to the skilled worker and are described for example in Schüler, D., and Köhler, M. (1992) “The isolation of a new magnetic spirillum” Zentralbl. Mikrobiol. 147: 150-151, Bazylinski, D. A., Frankel, R. B., and Jannasch, H. W. (1988) “Anaerobic magnetite production by a marine, magnetotactic bacterium” Nature 334: 518-519, Kawaguchi, R., Burgess, J. G., and Matsunaga, T. (1992) “Phylogeny and 16s rRNA sequence of Magnetospirillum sp. AMB-1, an aerobic magnetic bacterium” Nucleic. Acids. Res. 20: 1140, Meldrum, F. C., Mann, S., Heywood, B. R., Frankel, R. B., and Bazylinski, D. A. (1993) “Electron-microscopy study of magnetosomes in a cultured coccoid magnetotactic bacterium” P. Roy. Soc. Lond. B. Bio. 251: 231-236, Meldrum, F. C., Mann, S., Heywood, B. R., Frankel, R. B., and Bazylinski, D. A. (1993) “Electron-microscopy study of magnetosomes in 2 cultured vibrioid magnetotactic bacteria” P. Roy. Soc. Lond. B. Bio. 251: 237-242, and Schleifer, K., Schüler, D., Spring, S., Weizenegger, M., Amann, R., Ludwig, W. and Köhler, M. (1991) “The genus Magnetospirillum gen. nov., description of Magnetospirillum gryphiswaldense sp. nov. and transfer of Aquaspirillum magnetotacticum to Magnetospirillum magnetotacticum comb. nov.” Syst. Appl. Microbiol. 14: 379-385, all of which are incorporated herein by reference.

The magnetic nanoparticles of the invention have been in use in the diagnostics of pathological processes for some years now. Their use as contrast medium in magnetic resonance tomography (MRT) is of great importance. MRT contrast media for liver and spleen which have been approved up to now are available under the trademarks Endorem® or Resovist® and can be used for successful imaging of hepatic metastases and lowly differentiated hepatic tumors which lack macrophages. Resovist® is a ferrofluid with a hydrodynamic diameter of approx. 60 nm and a core diameter of from 3 nm to 15 nm. By now, newer ferrofluids are available which consist of larger particles having hydrodynamic diameters of from 120 nm to 150 nm.

Particular preference is given to the biocompatible, magnetic nanoparticles of the invention being absorbed by the cells.

According to the invention, the biocompatible, magnetic nanoparticles are used for the targeted movement of migrating cancer cells in an external magnetic field (magnetotaxis), in order to make said cells accessible as a collective to surgical intervention or hyperthermia.

One possibility of administering magnetic nanoparticles is that of direct application to the brain in order to avoid the nanoparticles being held up by the blood brain barrier. After administration, the particle-loaded cells may then be directed to the target region by applying an external magnetic field.

Another, preferred possibility is that of coupling to the nanoparticles specific antibodies which bind to antigens in the affected region. Preferably, the antibodies bind specifically to surface antigens of glioblastoma cells, without healthy tissue being affected.

1. A biocompatible, magnetic nanoparticle for use in the therapy of glioblastomas in a static magnetic field, wherein the biocompatible, magnetic nanoparticles are used for the targeted movement of migrating cancer cells in an external magnetic field (magnetotaxis), in order to make said cells accessible as a collective to surgical intervention or hyperthermia.

2. The nanoparticle as claimed in claim 1, characterized in that it has a hydrodynamic size of less than 1 μm.

3. The nanoparticle as claimed in claim 1, characterized in that it has a core diameter of from 1 nm to 300 nm.

4. The nanoparticle as claimed in claim 1, characterized in that it is selected from iron (Fe), gadolinium (Gd) or the oxides thereof.

5. The nanoparticle as claimed in claim 4, characterized in that it is selected from magnetite or maghemite.

6. The nanoparticle as claimed in claim 1, characterized in that it has a shell material.

7. The nanoparticle as claimed in claim 6, characterized in that the shell material is selected from polymers selected from the group consisting of dextran, carboxydextran, polyethylene glycol, starch, albumin, and a biomimetic material selected from the group consisting of lipids, fatty acids and citrate.

8. The nanoparticle as claimed in claim 7, characterized in that the shell material additionally includes antibodies.

9. The nanoparticle as claimed in claim 8, characterized in that the antibody or antibodies specifically binds/bind to surface antigens of glioblastoma cells.

10. The nanoparticle as claimed in claim 1, characterized in that the nanoparticles are magnetosomes from magneto static bacteria.

11. The nanoparticle as claimed in claim 10, characterized in that the magnetosomes are obtainable from Magnetospirillum gryphiswaldense MSR-1, Magnetospirillium magnetotacticum, Magnetospirillium spec. AMB-1, magnetic coccus MC-1 or magnetic Vibrio MC-1.

12. The nanoparticle of claim 2, wherein the nanoparticle has a hydrodynamic site in the range of 5 nm to 300 nm.

13. The nanoparticle of claim 12, wherein the nanoparticle has a hydrodynamic site in the range of 50 nm to 200 nm.

14. The nanoparticle of claim 3, wherein the nanoparticle has a core diameter of 3 nm to 50 nm.

15. A method for the treatment of glioblastomae comprising incorporating biocompatible, magnetic nanoparticles into the brain of a patient, and subjecting the patient to an external static magnetic field to provide a targeted movement of migrating cancer cells in said external magnetic field (magnetotaxis), and wherein said cells are subsequently collectively subjected to surgical intervention or hyperthermia.

16. The method of claim 15, wherein said biocompatible, magnetic nanoparticles have a hydrodynamic size of less than 1 μm.

17. The method of claim 15, wherein said biocompatible, magnetic nanoparticles have a core diameter of from 1 nm to 300 nm.

18. The method of claim 15, wherein said biocompatible, magnetic nanoparticles are selected from the group consisting of iron (Fe), gadolinium (Gd) and the oxides thereof.

19. The method of claim 18, wherein said biocompatible, magnetic nanoparticles are selected from the group consisting of magnetite or maghemite.

20. The method of claim 1, wherein said biocompatible, magnetic nanoparticles have a shell material.

21. The method of claim 6, wherein the shell material is a polymer selected from a group consisting of dextran, carboxydextran, polyethylene glycol, starch, albumin, and a biomimetic material.

22. The method of claim 21, wherein the shell material is a biometric material selected from the group consisting of citrate.
